(19) United States
(12) Patent Application Publication
(10) Pub. No.: US 2016/0139049 A1
(43) Pub. Date: May 19, 2016

(54) OPTICAL SENSOR AND MEASURING APPARATUS FOR QUANTITATIVELY DETECTING AN ANALYTE IN A SAMPLE

(71) Applicant: PreSens Precision Sensing GmbH, Regensburg (DE)

(72) Inventors: Daniel Riechers, Regensburg (DE); Athanasios Apostolidis, Regensburg (DE)

(21) Appl. No.: 15/005,664

(22) Filed: Jan. 25, 2016

Related U.S. Application Data
(63) Continuation of application No. PCT/IB2014/063620, filed on Aug. 1, 2014.

(30) Foreign Application Priority Data
Aug. 9, 2013 (DE) DE102013108659.4

(51) Int. Cl. G01N 21/64; G01N 33/00

(52) U.S. Cl. CPC G01N 21/643; G01N 33/0062; G01N 33/0073; G01N 2021/6439

(57) ABSTRACT

A sensor for quantitatively detecting an analyte in a sample, wherein an optical behavior of at least one dye depending on the analyte is used for the quantitative detection is provided. The at least one dye is contained in a medium. A restrictor is provided in order to mechanically limit volume changes of the medium. Furthermore, an osmolality in the medium is set in such a way that the osmolality is higher than the highest sample osmolality for which the sensor is to be used. The cooperation of the restrictor with the specified high osmolality results in a greatly reduced osmolality cross-sensitivity of the sensor. The restrictor can be embedded into the medium as a membrane, mat, braided material, woven fabric, or mesh. Alternatively, the restrictor can also include a carrier plate having a plurality of recesses, in which the medium is arranged. A corresponding measuring apparatus is also provided.

This is a Continuation of International Patent Application No. PCT/M2014/063620, filed Aug. 1, 2014 which claims the benefit of German Patent Application DE 10 2013 108 659.4, filed Aug. 9, 2013, both of which are hereby incorporated by reference herein.

### BACKGROUND

The invention relates to a sensor for quantitatively detecting an analyte in a sample, wherein an optical behavior of at least one dye is used for quantitatively detecting the analyte.

The translation DE69430003T2 of the European patent EP 0 731 664 B1 issued for international application PCT/US94/12146, published as WO95/15114, discloses a sensor with an indicator dye contained in an aqueous phase. The dye can be contained in a liquid adsorbed to particles or absorbed in particles, the particles in turn being embedded into a polymer material permeable to gas and light, which does not let pass liquid water. The aqueous phase may also be enclosed in microcavities. Furthermore, the aqueous phase may contain a buffer, as well as salts for setting a desired osmolarity.

The international publication WO 00/26655 A1 of the international application PCT/US99/25506 discloses a dye enclosed in a recess of a sensor membrane. A perforated metal disc is arranged below the sensor membrane, in order to prevent a swelling of the dye layer.

The German translation DE 69219061 T2 of the European patent EP 0 539 175 B1 discloses, amongst other things, the impregnation of a carrier with a mixture of reagents. This contains a buffer, and may contain for example cellulose, gum arabic, or polyvinylpyrrolidone as a binder.

The German translation DE 69612017 T2 of the European patent EP 0 873 517 B1 issued on international application PCT/US96/16469, published as WO97/19348, discloses an aqueous phase with indicator and buffer, which is located within a second hydrophobic phase. This emulsion may contain a humectant. The aqueous phase is located within microcompartments. Substances may be added which regulate the osmotic pressure.

The German published patent application DE 2 134 910 relates to a dye for dyeing white blood cells, as well as to a method for analytically determining white blood cells in the blood. An aqueous solution with a dye is mixed with a blood sample. Therein the aqueous solution contains additives for keeping a pH-value and an osmolality within the normal range for human blood plasma.

The international publication WO 2009/140559 A1 of international application PCT/US2009/044048 discloses a sensor element of layered structure. An indicator is bound to a porous support membrane arranged on a polymer substrate. The support membrane may be made of a woven plastic.

The US publication US 2004/171094 A1 relates to a dye with analyte-dependent phosphorescence enclosed in hydrophobic polymer particles. The particles may in turn be embedded in a layer or matrix, which may be absorbent to water and may swell, when water is absorbed.

The US publication US 2008/215254 A1 discloses sensors comprising one or plural layers arranged on a transparent carrier. The layers may be made of a polymer, and may for example be arranged in the wells of a microtiter plate or at the end of an optical fiber.

### SUMMARY OF THE INVENTION

Optical sensors for detecting acidic or basic gases, for example ammonia or sulphur dioxide, in gaseous samples or in solution in a liquid, are widely known. These sensors contain a dye with an optical behavior which is affected by the gas to be detected, often indirectly, for example via a change of the pH-value of a buffer solution containing the dye. Usually the buffer solution with the dye is separated from the sample to be analyzed by a gas permeable material, for example a gas permeable polymer. If water from the sample enters the buffer solution, because the gas permeable material is itself water absorbent, and because water can then reach the buffer solution from the gas permeable material, or because water in the form of vapor diffuses through the gas permeable material into the buffer solution, the concentration of the buffer and thus the pH-value changes. In this way conditions result in the sensor which do no longer correspond to the calibration of the sensor. As the transport of water into the buffer solution is determined by the osmolalities of the buffer solution and the sample, a cross-sensitivity to osmolality of the sensor results. This is particularly problematic, if the osmolality of the sample changes over the course of a measurement. This problem for example arises in the biotechnology sector, where sensors of the kind described are used for monitoring bio-processes, such as fermentations or cell cultivations. In the medical sector, too, at online or offline measurements in blood, urine, or tissues, large changes of the osmolality can occur, which, with the prior art sensors described, lead to large measurement errors.

It is an object of the invention to provide a sensor which shows a cross-sensitivity to osmolality lower than in the prior art, and the calibration of which is largely independent of the osmolality of the sample.

It is a further object of the invention to provide a measuring apparatus capable of quantitatively detecting an analyte in a sample, without requiring the effort to take into account various possible osmolalities of a sample for calibration of the measuring apparatus and when taking measurements with the measuring apparatus.

The present invention provides a sensor for quantitatively detecting an analyte in a sample, the sensor including at least one dye having an optical behavior which, within the sensor, can be affected by the analyte, which thus depends directly, or, due to the configuration of the sensor, indirectly, on the analyte. This means that from the optical behavior of the at least one dye, quantitative conclusions with regard to the analyte can be inferred, for example a concentration or a partial pressure for the analyte can be determined. To this end preferentially a calibration of the sensor is used. Quantitatively detecting the analyte is understood as determining a value for the concentration or the partial pressure of the analyte up to error bounds known in the art, but also as only finding that the concentration or the partial pressure of the analyte are within a pre-determined range; the pre-determined range therein may have a lower bound and an upper bound, or either only a lower bound or only an upper bound.

Furthermore the sensor according to the invention includes a medium, which contains the at least one dye, and also includes a restriction means, by which a volume change of the medium is mechanically limited. The medium may for example include a liquid in which the at least one dye is contained in solution; depending on the embodiment, the medium may contain further components, as will be discussed below.

According to the invention an osmolality in the medium is higher than a pre-defined maximum sample osmolality for which a use of the sensor is intended. For samples with an osmolality higher than this maximum sample osmolality the sensor according to the invention should not be used, as in such a case the results of the measurements would not be reliable.

From the combination of the defined osmolality in the medium and the mechanical limitation of the volume change of the medium a defined absorption of solvent, for example water, into the medium results, when the sensor is brought into contact with a sample, in which the analyte, i.e. the substance to be detected, is in solution in the solvent. In the case of gaseous samples, water can enter into the sensor as water vapor contained in the sample, and analogously a defined absorption of water results. As long as the osmolality in the medium is higher than in the sample, the osmotic pressure is working towards an influx of solvent into the medium. This influx of solvent would cause a swelling of the medium. By the restriction means, however, a change of volume of the medium, and thus in particular a swelling, is sharply limited; therefore also the absorption of solvent into the medium is clearly limited. As a result, for a given sensor there is a defined absorption of solvent into the medium, which is independent of the osmolality of the sample, as long as the osmolality of the sample is lower than the osmolality in the medium. This defined absorption of solvent can be taken into account when calibrating the sensor. In this way a calibration of the sensor becomes independent of the osmolality of the sample in which the sensor is used, as long as the osmolality of the sample is lower than the osmolality in the medium.

In some embodiments the medium includes a carrier material, which for example may be a polymer. One effect of the carrier material is to fix the spatial distribution of the at least one dye or of particles containing the dye in the medium.

In particular embodiments the at least one dye is contained in cavities within the carrier material. Therein these cavities may be formed by the carrier material itself and enclose the at least one dye. The carrier material may also be porous, and the at least one dye may be located within the pores of the carrier material, which at least partially are in communication with each other. In particular, the at least one dye may also be adsorbed to the interior walls of the pores.

In other embodiments the cavities are enclosed by shells different from the carrier material. In particular, the shells may be made of a material different from the carrier material. For example, hollow particles may be distributed within the carrier material, the interior of the hollow particles containing the at least one dye.

In particular embodiments the cavities are micelles, and the at least one dye is located in the interior of the micelles.

In other embodiments the at least one dye is homogeneously mixed with the carrier material and fixed within the carrier material.

The mechanical limitation of a volume change of the medium in one embodiment is achieved by the medium being arranged in a plurality of recesses formed in a carrier plate. The carrier plate therein may for example be made of a glass or a plastic, for instance polycarbonate or polyethylene terephthalate (PETE). Carrier plates of metal, for example stainless steel, are also possible. In case the medium contains a carrier material, the material of the carrier plate should have an elastic modulus equal to at least a hundred times the elastic modulus of the carrier material, in order to achieve a significant limitation of the volume change. It is further advantageous with respect to an efficient limitation of the volume change, if the filling height of a recess with carrier material is at least twice a diameter of the recess. Limiting the volume change can be improved further, if the carrier material sticks to the interior wall of the recess. If there is no carrier material, the medium should be enclosed completely in the recesses; this can for example be accomplished by cover layers which close the recesses.

In some embodiments the recesses reach through the carrier plate entirely, i.e. are holes in the carrier plate.

In another embodiment, in which the medium includes a carrier material, the mechanical limitation of a volume change of the medium is achieved by embedding, into the carrier material, a membrane, a mat, a braided material, a woven fabric, or a mesh constituting the restriction means. A carrier material sticking to the restriction means therein is an advantage. The restriction means is made of material with high tensile stiffness, which is to be understood as an elastic modulus of the restriction means being at least a hundred times larger than an elastic modulus of the carrier material.

In a further embodiment of the sensor according to the invention a shell partially encloses the medium. The medium therein includes a carrier material, the shell is the restriction means.

In a further embodiment the medium is completely enclosed by a shell. Here, too, the shell is the restriction means. A carrier material may be included in the medium; however, embodiments without a carrier material may be used, too.

In a further development of the preceding cases, a shell, which is the restriction means, forms plural cavities, fully or partially enclosed by the material of the shell. Each cavity contains a medium with a dye. Therein at least two of the dyes used are different from each other. The dyes may for example react to different analytes, and thus provide information on different analytes from the respective location of the sensor.

The shell forming the restriction means in the preceding embodiments must have a sufficient mechanical tensile stiffness in order to provide sufficient mechanical resistance to a change of volume of the medium. Suitable materials for the shell for example are polyvinylidene fluoride (PVDF), Teflon, polyether sulfone, polystyrene, silicon dioxide, or ormosils; however, the possible materials are not limited to the ones listed here.

In an embodiment of the sensor according to the invention the at least one dye is mixed with a buffer solution, which then is to be considered part of the medium. This is in particular the case with sensors the sensor effect of which is based on a pH-value dependent optical behavior of the at least one dye, and where the analyte causes a change of the pH-value in the medium.

The osmolality in the medium may be set by adding at least one substance to the medium when manufacturing the sensor. In embodiments the at least one substance may be mixed with the at least one dye. Substances used for setting the osmolality in the medium may for example be salts, like NaCl or KCl, polyelectrolytes, or neutral molecules like for example sugar, such as glucose, fructose, mannose, sucrose. These added substances, too, are to be considered part of the medium. Herein it is of course important for the added substances not to disturb the quantitative detection of the analyte.

In a particular embodiment the sensor includes a hygroscopic substance. Such embodiments may also be used in gaseous samples, like the atmosphere. The hygroscopic substance absorbs water from the sample, which in the sample is present for example as water vapor. In this way an aqueous environment is created for the at least one dye. Therefore dyes and additives like for example buffer can be used, which otherwise are limited to aqueous samples, for measuring in the gas phase. Advantageously the hygroscopic substance is mixed with the at least one dye.

The optical behavior of the dye which is used for quantitatively detecting an analyte may for example be a luminescence, wherein luminescence at least comprises phosphorescence and fluorescence. Likewise a reflection of light or an absorption of light may be used for quantitatively detecting the analyte. A further possibility is using a color of the dye. Therein the color of the dye, the reflection or absorption of light, or the luminescence exhibit a dependence on the analyte. This dependence, in case of a luminescence, may comprise that a relaxation time of the luminescence, which may be a relaxation time for the intensity of the luminescence or for a polarization of the luminescence, depends on the analyte. It is also possible that intensity or wavelength of the luminescence light depend on the analyte. In case of a color, the dye may assume a different color according to concentration or partial pressure of the analyte. In case of reflection or absorption of light, the reflectivity or the degree of absorption, respectively, of a layer containing the dye for certain wavelengths of light may change in dependence on the analyte. Also, more than one type of optical behavior may be used for measuring, for example a relaxation time of the luminescence and an absorption behavior. To this end more than one dye may be used, so that for example the luminescence behavior of a first dye and the absorption behavior of a second dye are evaluated, in order to quantitatively determine an analyte. If more than one dye is used, also the efficiency of non-radiative energy transfer between the dyes, for example Förster resonance energy transfer, may be used for quantitatively determining the analyte, to the extent that this efficiency quantitatively depends on the analyte.

The dependence of the optical behavior on the analyte may result from a direct interaction between the analyte and the at least one dye, for example an exchange of energy or a chemical reaction between molecules of the dye and of the analyte, or from an indirect interaction via substances added to the dye. A general prerequisite for the correct operation of the sensor therefore is that the analyte can engage in such a direct or indirect interaction with the at least one dye. For example, if, in a sensor according to the invention, dye and buffer solution are located in cavities within a carrier material, or are enclosed by the restriction means, the analyte must be able to diffuse through the carrier material or the restriction means, respectively, in order to reach the mixture of dye and buffer solution.

Examples of analytes are gases in gaseous mixtures or gases in solution in liquids. For example, gas in solution in water, such as sulphur dioxide, carbon dioxide, carbon monoxide, or ammonia, may be captured by a sensor according to the invention. Ammonia, showing a basic reaction in water, is an example where a dye with a pH-value dependent optical behavior may be selected as dye for the sensor. In dependence on the concentration of the ammonia in solution in the sample a pH-value results in the medium which can be determined from the optical behavior of the dye. Indirect conclusions on the concentration of ammonia are possible in this way.

Of course, a direct calibration of the concentration of ammonia to the optical behavior is possible, an explicit determination of a pH-value is not required then. The just described example of ammonia detection also is an example for an indirect interaction between the dye and the analyte, here the ammonia. The interaction here is via a buffer solution mixed with the dye, by the analyte changing the pH-value of the buffer solution, and the optical behavior of the dye depending on the pH-value of its environment, here of the buffer solution. Analogous statements apply to sulphur dioxide and further gases. An example of a dye with a pH-value dependent fluorescence behavior, with which carbon dioxide can be detected, is hydroxypyrenetrisulfonic acid (HPTA). For measuring sulphur dioxide, for example bromocresol purple may be used. For quantitatively detecting ammonia, bromothymol blue or bromophenol blue may be used. Many further dyes and their possible uses for quantitatively detecting diverse substances are sufficiently well known to the person skilled in the art.

Sensors according to the invention may, however, also be used for quantitatively detecting other substances in solution, including ions.

The measuring apparatus according to the invention for quantitatively detecting an analyte in a sample includes a sensor according to the invention as described above. A control and evaluation unit is provided for quantitatively determining the analyte from the optical behavior of the at least one dye of the sensor. Furthermore, the measuring apparatus includes optics which is configured to guide light from the control and evaluation unit to the sensor, and likewise to guide light from the sensor to the control and evaluation unit, in order to in this way capture the optical behavior of the at least one dye, from which the analyte then is quantitatively determined. The light guided from the control and evaluation unit to the sensor may, depending on the configuration of the sensor, be excitation light for a luminescence of the at least one dye, or light with which a color, a reflectivity, or a degree of absorption of a layer containing the at least one dye in the sensor is determined. The control and evaluation unit therein controls the light emitted by it, for example defined sequences of light pulses or light signals of modulated intensity may be generated. In embodiments of the measuring apparatus the optics is free beam optics. In other embodiments of the measuring apparatus the optics is fiber optics.

By using a sensor according to the invention it is possible to quantitatively determine an analyte in a sample, without having to take into account laboriously various possible sample osmolalities when calibrating the measuring apparatus and when performing measurements with the measuring apparatus, as the calibration of a sensor according to the invention is independent of the sample osmolality, as has been described above. Cross-sensitivities to osmolality therefore neither exist for the sensor nor for the measuring apparatus.

It is possible for the sensor, for example of platelet-shape, to be arranged at an end of the optical fiber, and to be inserted into the sample with this end of the optical fiber. However, it is also possible for the sensor to be, for example, arranged in the interior of a sample container at a wall of the sample container. The wall therein is transparent for the light guided from the control and evaluation unit to the sensor, and also for the luminescence light emitted from the sensor, or the light reflected or backscattered from the sensor. The light therein may be guided by free beam optics. Likewise, the light may be guided by an optical fiber, wherein the optical fiber runs to a corresponding location at the outside of the wall and ends there; in this case the light exits from the optical fiber there, passes through the wall of the sample container transparent to it, and impinges on the sensor. Light from the sensor passes along the inverse path. A further possibility is for one or plural sensors according to the invention to be dispersed within a sample, for example to float in a liquid sample. The optical behavior of the dyes in the sensors may then for example be captured by a camera, which in this case is a part of the control and evaluation unit.

As already mentioned, advantageously a calibration of the sensor is used for quantitatively determining the analyte. To this end corresponding calibration data for the sensor may be provided in the control and evaluation unit, for example electronically stored.

It should be mentioned that for explicitly quantitatively determining the analyte from the optical behavior of the at least one dye via a corresponding calibration various possibilities are known. For example, a relaxation time of a luminescence of the at least one dye may be calibrated against partial pressure or concentration of the analyte. Instead of using the relaxation time itself, quantities dependent thereon may be used, which may be determined experimentally more easily and more directly, for example ratios of integrals of the time-dependence of luminescence signals, or phase shifts between a modulated excitation signal and the luminescence response of the dye. These and further possibilities have been sufficiently described in the prior art, for example in German patent application DE 10 2011 055 272 A1 and the art cited therein.

### BRIEF DESCRIPTION OF THE DRAWINGS

Below the invention will be further described by embodiments and the accompanying schematic figures.

FIG. 1 shows a sensor according to the invention, wherein the restriction means is a carrier plate with a plurality of recesses.

FIG. 2 shows a cross section of a recess of the carrier plate shown in FIG. 1.

FIG. 3 shows a sensor, wherein the restriction means is a braided material.

FIG. 4 shows a plurality of cavities in the carrier material of a sensor according to the invention.

FIG. 5 shows a variant of the configuration shown in FIG. 4.

FIG. 6 shows a further cross section of a sensor with carrier plate.

FIG. 7 shows a cross section of a sensor with a completely perforated carrier plate.

FIG. 8 shows a sensor according to the invention, wherein the medium is completely enclosed by the restriction means.

FIG. 9 shows a sensor according to the invention, wherein the medium is arranged in more than one cavity and completely enclosed by the restriction means.

FIG. 10 schematically shows a measuring apparatus according to the invention, wherein the sensor according to the invention is used.

FIG. 11 schematically shows a further variant of a measuring apparatus according to the invention, wherein the sensor according to the invention is used.

FIG. 12 schematically shows a further variant of a measuring apparatus according to the invention, wherein the sensor according to the invention is used.

### DETAILED DESCRIPTION

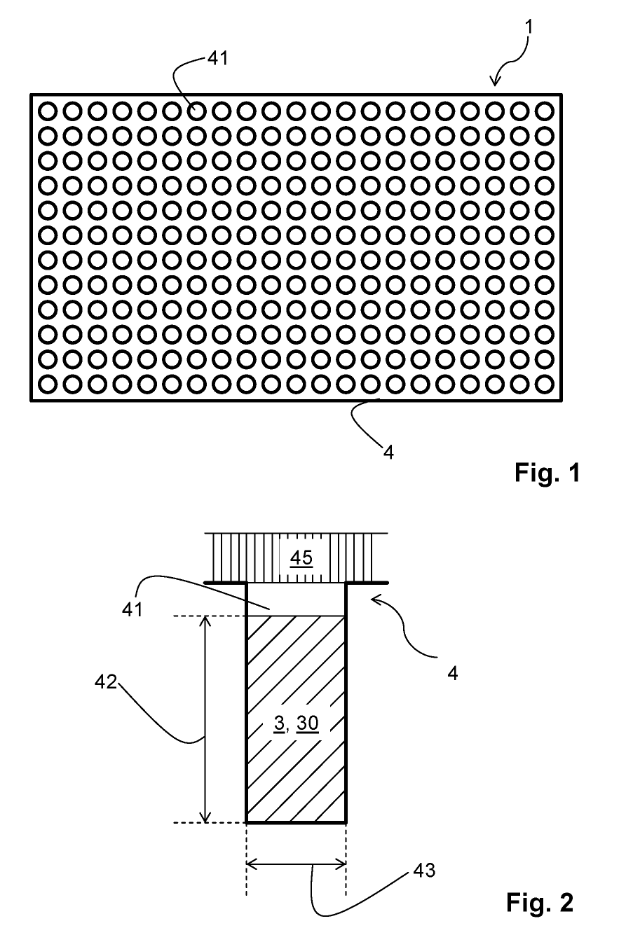

FIG. 1 shows a possible embodiment of a sensor 1 according to the invention. A plurality of recesses 41 are formed in a carrier plate 4. When manufacturing the sensor 1, for example a sensor mixture of the at least one dye and a medium, which contains buffer solution and/or substances, such as salts, polyelectrolytes, or sugar, for setting the osmolality, as well as non-polymerized carrier material, may be applied on the carrier plate 4 with a blade. The applied sensor mixture is sucked into the recesses 41 by subsequent exposure of the carrier plate 4 to vacuum. Alternatively, the applied sensor mixture may be transferred into the recesses 41 for example by centrifugation of the carrier plate 4 with the sensor mixture applied. Surplus sensor mixture is removed, and the sensor mixture in the recesses polymerized, for example thermally or induced by light.

The rectangular shape of the sensor 1 shown is not a limitation of the invention. However, for manufacturing sensors according to the invention, large carrier plates may be provided with a sensor mixture in the manner described above, and then sensors of the desired size and shape may be sawed, cut, punched, or otherwise separated from the carrier plate. To this end also predetermined breaking points may be provided in the carrier plate.

The circular cross section of the recesses 41 shown is not a limitation of the invention. Different cross sections are also possible, for example rectangular or hexagonal; in the hexagonal case in particular a honeycomb structure of the carrier plate 4 is conceivable.

Typical diameters of the recesses 41 are 10 to 500 micrometers. Typical depths of the recesses 41 are 100 to 500 micrometers. The material thickness of the carrier plate then advantageously ranges between 100 micrometers and 1 millimeter. These dimensions, however, are not a limitation of the invention. Diameters from the nanometer range to the centimeter range and beyond are also conceivable.

FIG. 2 shows a cross section of a recess 41 in a carrier plate 4 as shown in FIG. 1. The recess 41 is filled with a medium 3 which contains carrier material 30 and the at least one dye (not shown here). In the example shown a filling height 42 of the recess 41 with the medium 3 is larger than twice a diameter 43 of the recess 41. As already mentioned above, such a choice of dimensions contributes to a limitation of a change of volume, in particular of a swelling, of the medium 3.

Further shown is an optional cover layer 45, which covers the recesses 41. If, while measuring, the cover layer 45 is towards a sample to be examined, the cover layer 45 advantageously is permeable for the analyte. The material of the carrier plate 4 then advantageously is transparent, the optical behavior of the dye may then be captured through the material of the carrier plate 4. In this case, the cover layer 45 may be reflecting, which facilitates capturing the optical behavior of the dye, in particular, if the optical behavior is a luminescence phenomenon. Likewise, the material of the carrier plate 4 may be permeable to the analyte. In this case, when measuring, the carrier plate 4 would be towards the sample to be examined. The cover layer 45 then preferentially is transparent, the optical behavior of the dye may then be captured through the material of the cover layer 45.

One possibility to obtain a reflecting cover layer 45 is to apply a non-polymerized layer containing titanium dioxide particles to the carrier plate 4 with a blade, and then to polymerize the applied layer. A non-transparent cover layer 45 may for example be a film of PVDF or Teflon. The film may for example be glued onto the carrier plate 4 or thermally fused thereto. Such a film is permeable to gases. Alternatively, also the carrier plate 4 may be made of PVDF or Teflon, so that in this case the carrier plate is permeable to gases.

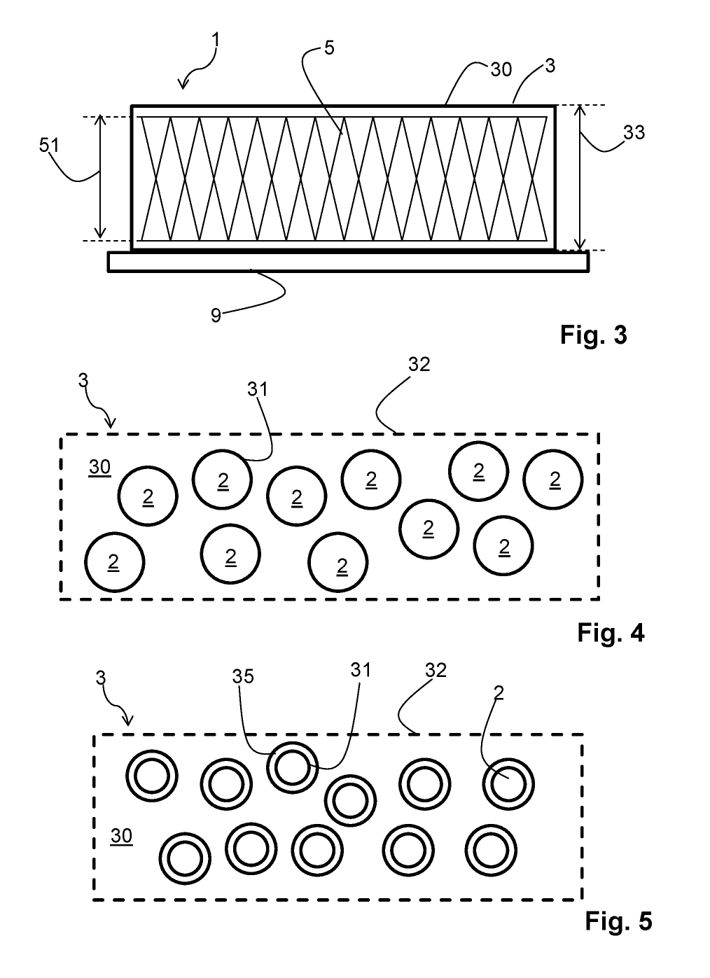

FIG. 3 shows an embodiment of the sensor 1 according to the invention, wherein the restriction means is a braided material 5. The braided material 5 is embedded into the carrier material 30, which is part of the medium 3; the medium 3 includes the at least one dye and, as the case may be, further additives, which are not shown here. In the example shown, the medium 3 is applied on a support plate 9. The support plate 9 preferentially is transparent. In this way the medium 3 with the at least one dye may be brought into contact with a sample, and the optical behavior of the dye may be captured through the transparent support plate 9. Here, too, analogously to FIG. 2, a reflecting cover layer may be provided. In the example discussed, this would have to be provided parallel to the support plate 9, at the side of the medium 3 opposite the support plate 9, and would have to be permeable to the analyte. Alternatively, the support plate 9 may be reflecting instead of transparent. The optical behavior of the dye would then be captured from the side of the sensor 1 opposite the support plate 9, and the support plate 9 would have to be permeable for the analyte.

A sensor 1 of this kind may for example be manufactured by applying a sensor mixture of the kind already mentioned onto the support plate 9 with a blade, and then placing the braided material 5 onto the sensor mixture. The non-polymerized sensor mixture will enclose the braided material 5, so that eventually the braided material 5 is embedded in the medium 3, and thus in particular in the carrier material 30. Then the carrier material 30 may be polymerized, for example thermally or induced by light.

A typical thickness of the applied layer of sensor mixture is between 10 and 1000 micrometers. A thickness 51 of the restriction means 5 advantageously is calculated as the thickness of the applied layer divided by (1−porosity of restriction means). An overall thickness 33 of the applied layer of medium 3 with embedded restriction means 5 results.

Instead of a braided material also a membrane, a mat, a woven fabric, or a mesh may be used. Specifically a PETE-mat (e.g. Freudenberg, novatexx 2481) may be used. In case of a mat or a woven fabric the sensor mixture may be absorbed by capillary effects into mat or woven fabric and impregnate it. Therein, advantageously, mat or woven fabric are oleophilic. An attachment of a polymer carrier material to the restriction means forming a mat, woven fabric, braided material, mesh, or membrane may be improved by activating the restriction means by plasma treatment, corona treatment, or by a primer.

FIG. 4 shows a region 32 of the medium 3. The medium 3 contains the at least one dye 2 in cavities 31 formed within the carrier material 30. Besides the dye 2, the cavities 31 may, depending on the embodiment of the sensor 1, also contain a buffer solution, a hygroscopic substance, a substance for setting the osmolality in the medium 3, or further substances. The osmolality set in the medium 3 in the embodiment shown refers to the osmolality within the cavities 31.

A medium 3 of this configuration may be used both with sensors 1, in which the medium 3 is arranged in recesses 41 of a carrier plate 4, as shown in FIGS. 1 and 2, and with sensors 1 having the restriction means embedded into the medium 3, as shown in FIG. 3.

The cavities 31 may for example be micelles. A carrier material 3 of this structure may for example be obtained by emulsifying a micelle-forming mixture of dye, buffer, and, as the case may be, further substances, in a silicone monomer, arranging the silicone monomer in the recesses 41 of a carrier plate 4, or embedding a restriction means in the silicone monomer, and subsequently cross-linking the silicone monomer.

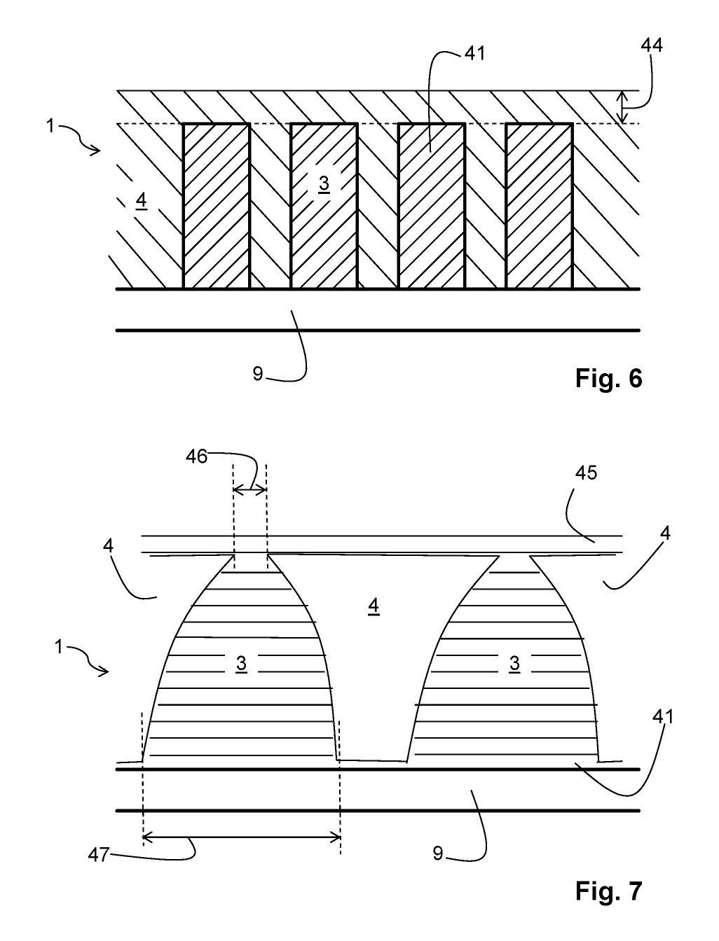

FIG. 5 is largely similar to FIG. 4. However, the cavities 31 of the configuration shown in FIG. 5 are enclosed by shells 35, which are different from the carrier material 30. For example, the cavities 31 may be the interior of nanoparticles immobilized in the carrier material 30.

FIG. 6 shows a cross section of a further embodiment of a sensor according to the invention with a carrier plate 4. The carrier plate 4 is made of a polymer permeable to carbon dioxide, with recesses 41 formed in the polymer by embossing. The recesses 41 contain the medium 3. After filling of the recesses 41 with the medium 3 the carrier plate 4 is glued to a transparent support plate 9, so that the support plate 9 closes the openings of the recesses 41. The medium 3 thus is enclosed in the recesses 41. Here the medium 3 may be used without carrier material, but, for example, also a medium 3 is possible which is configured as described in FIG. 4 or FIG. 5. In the embodiment shown, when measuring, the support plate 9 is facing away from the sample. Towards the sample there remains a portion of the polymer of the carrier plate 4, with a thickness 44, which, for example, is between 1 and 50 micrometers. The material of the carrier plate may act as optical insulation, the portion of thickness 44 may be reflecting.

FIG. 7 shows an embodiment of the sensor according to the invention with carrier plate 4, wherein the carrier plate 4 is completely perforated by the recesses 41. The carrier plate 4 may for example be made of stainless steel (e.g. 1.4401, 1.4435, 1.4571), in which case the recesses 41 may for example be created by etching. The recesses 41 are filled with the medium 3. The carrier plate 4, after filling the recesses 41 with the medium 3, is glued to a transparent support plate 9, so that the support plate 9 closes openings of the recesses 41 on one side of the carrier plate 4. At the opposite side of the carrier plate 4 the openings of the recesses 41 are closed by a layer 45. The layer 45 may for example be a silicone layer containing titanium dioxide and therefore be reflecting; PVDF also is a possible material for the layer 45. When measuring, the layer 45 would be towards the sample.

In the embodiment shown the recesses 41 are asymmetric in the sense that on the sample side the openings of the recesses, i.e. the openings covered by layer 45, have a diameter 46 which is smaller than a diameter 47 of the openings covered by the support plate 9. It has turned out that with such an asymmetric geometry the cross-sensitivity to osmolality is further reduced in comparison with a symmetric geometry like in FIG. 2; however, the response time of the sensor is longer in comparison.

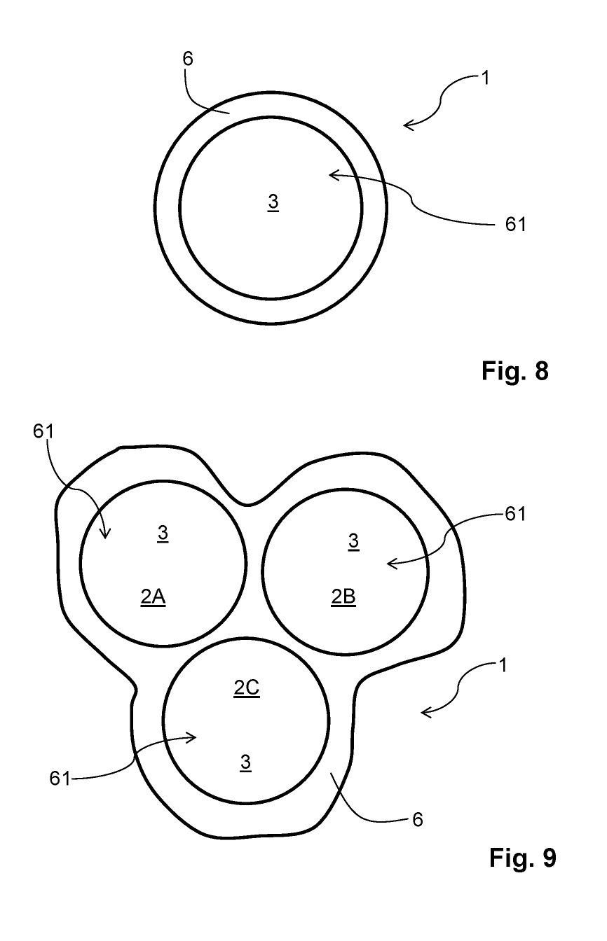

FIG. 8 shows a sensor 1 according to the invention, wherein the medium 3 is completely enclosed by a shell 6. The medium is arranged in a cavity 61 enclosed by the shell 6. In this embodiment, the shell 6 is the restriction means. The shell 6 has to be both permeable to the analyte and transparent for the wavelengths of light occurring when capturing the optical behavior of the at least one dye. The medium 3 contains the at least one dye (not shown), and may further include for example a buffer solution and substances for setting the osmolality of the medium. A carrier material is not required; use of a carrier material is possible, however. In particular the medium 3 may also be structured as shown in FIG. 4 or FIG. 5. A sensor 1 of the kind shown here may be of spherical shape, which, however, is not a limitation of the invention. The diameter of such a sphere may be from the nanometer range up to for example 1 meter, or beyond. Such sensors for example may be used floating in a sample, for example in the interior of a bio-reactor, or in the interior of a cell.

Furthermore, it is possible to immobilize a plurality of such sensors 1 in an ambient matrix, for example in a polymer. The matrix therein must be permeable to the analyte.

FIG. 9 shows a further embodiment of a sensor 1 according to the invention. As in the embodiment of FIG. 8, a shell 6 is the restriction means. The shell, in this embodiment, encloses three cavities 61, in which, respectively, a medium 3 is located. In each cavity 61 there is a respective dye, 2A, 2B, and 2C. In the embodiment shown, the dyes 2A, 2B, and 2C are different from each other. Correspondingly, the media 3 in the respective cavities 61 may be different from each other, for example in order to be adapted to the respective dye 2A, 2B, or 2C. Embodiments with only two cavities 61 or with more than three cavities 61 are also possible.

If the optical behavior of the dyes 2A, 2B, 2C depends on respectively different analytes, a sensor of the type shown may be used to obtain information on these analytes from, within an accuracy defined by the dimensions of the sensor, the same location within a sample.

Apart from the sensors 1 shown in FIGS. 8 and 9, wherein the restriction means completely encloses the medium 3 as a shell 6, also sensors are conceivable, wherein the medium 3, which in this case advantageously includes a carrier material, is only partially enclosed by the restriction means.

A sensor as shown in FIG. 9 may also be obtained by connecting, for example gluing together or fusing together, plural, here specifically three, sensors of the type shown in FIG. 8, at their shells 6.

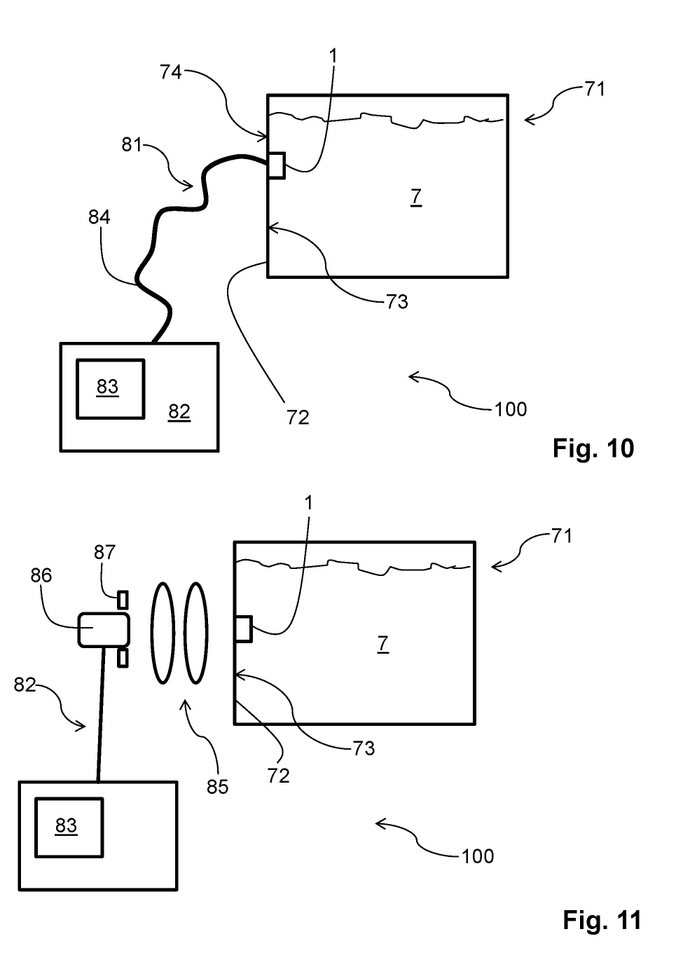

FIG. 10 shows a measuring apparatus 100 according to the invention, wherein a sensor 1 according to the invention is used. The sensor 1 is located within a sample container 71 in contact with an aqueous sample 7, and is fixed to an inner side 73 of a wall 72 of the sample container 71. In the example shown the optical behavior, depending on the analyte, of the dye of the sensor 1 is a luminescence behavior. Optics 81 for capturing the luminescence behavior includes an optical fiber 84. Via the optical fiber 84, excitation light may be guided to the sensor 1, in order to excite the luminescence of the dye. Likewise, luminescence light may be guided from the dye via the optical fiber 84 to the control and evaluation unit 82, which determines a concentration or a partial pressure of the analyte in the sample 7 from the luminescence light received. Herein, calibration data 83 provided for the sensor 1 are used. In the example shown, the optical fiber 84 ends at an outer side 74 of the wall 72 of the sample container 71. The wall 72 of the sample container 71 herein is transparent for the excitation light and the luminescence light. With optics 81 there are at least associated optical input coupling elements and output coupling elements for coupling in light into or coupling out light from the optical fiber 84. Such input coupling elements and output coupling elements are sufficiently well known to the skilled person and not shown here.

FIG. 11 is largely equal to FIG. 10, but as optics a free beam optics 85 is used here. Furthermore shown are a detector 86 for luminescence light and a ring light source 87 as light source for exciting the luminescence, which are part of the control and evaluation unit 82. The further elements have already been described in FIG. 10. Free beam optics 85 is shown only schematically, the free beam optics 85 may include further optical elements like filters and apertures besides lenses.

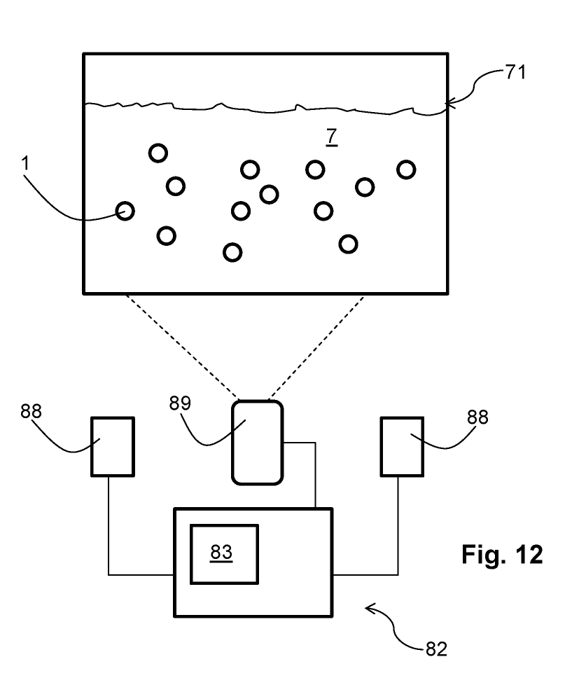

FIG. 12 shows a sample 7 in a sample container 71. A plurality of sensors 1 according to the invention, of the embodiment shown in FIG. 8 or FIG. 9, float in the liquid sample 7. For capturing the optical behavior of the at least one dye contained in the sensors 1, light sources 88 pertaining to the control and evaluation unit 82 and a camera 89, also pertaining to the control and evaluation unit 82, are provided. The control and evaluation unit 82 evaluates images recorded by the camera 89, and in this way can quantitatively determine the analyte in a space-resolved manner.

What is claimed is:

1. A sensor for quantitatively detecting an analyte in a sample, the sensor comprising:
at least one dye having an optical behavior affectable within the sensor by the analyte,
a medium containing the dye,
a restrictor, a volume change of the medium being mechanically limited by the restrictor, an osmolality in the medium being larger than a pre-defined maximum sample osmolality for which the sensor is provided.

2. The sensor as recited in claim 1 wherein the medium contains a carrier material.

3. The sensor as recited in claim 2 wherein the carrier material is a polymer.

4. The sensor as recited in claim 2 wherein the dye is contained in cavities within the carrier material.

5. The sensor as recited in claim 4 wherein the cavities are enclosed by shells different from the carrier material.

6. The sensor as recited in claim 4 wherein the cavities are formed by micelles.

7. The sensor as recited in claim 2 wherein the dye is homogeneously mixed with the carrier material.

8. The sensor as recited in claim 1 wherein the restrictor is a carrier plate with a plurality of recesses, the medium being arranged in the recesses.

9. The sensor as recited in claim 8 wherein a filling height of at least one recess of the recesses with the medium has at least twice the value of a diameter of the one recess.

10. The sensor as recited in claim 8 wherein at least one recess of the recesses perforates the carrier plate.

11. The sensor as recited in claim 2 wherein the restrictor is embedded into the carrier material, and the restrictor is a membrane, a mat, a braided material, a woven fabric, or a mesh.

12. The sensor as recited in claim 2 wherein the restrictor is a shell at least partially enclosing the medium.

13. The sensor as recited in claim 1 wherein the restrictor is a shell fully enclosing the medium.

14. The sensor as recited in claim 12 wherein plural cavities are formed by the shell, each cavity containing the dye, wherein at least two of the dyes differ from each other.

15. The sensor as recited in claim 13 wherein plural cavities are formed by the shell, each cavity containing the dye, wherein at least two of the dyes differ from each other.

16. The sensor as recited in claim 1 wherein the at least one dye is mixed with a buffer solution.

17. The sensor as recited in claim 1 wherein the osmolality in the medium is set by addition of at least one substance to the medium.

18. The sensor as recited in claim 1 wherein the sensor contains a hygroscopic substance.

19. The sensor as recited in claim 1 wherein the optical behavior of the at least one dye includes at least a luminescence or a color or a reflection of light or an absorption of light or a polarization.

20. A measuring apparatus for quantitatively detecting an analyte in a sample, wherein the measuring apparatus comprises:
a sensor for quantitatively detecting an analyte in a sample, the sensor comprising:
at least one dye having an optical behavior affectable within the sensor by the analyte,
a medium containing the dye,
a restrictor, a volume change of the medium being mechanically limited by the restrictor, an osmolality in the medium being larger than a pre-defined maximum sample osmolality for which the sensor is provided;
the measuring apparatus further comprising
a control and evaluation unit for quantitatively determining the analyte from the optical behavior of the at least one dye of the sensor; and
optics for guiding light between the control and evaluation unit and the sensor.

21. The measuring apparatus as recited in claim 20 wherein calibration data for the sensor are stored in the control and evaluation unit, the control and evaluation unit configured to take into account the calibration data when quantitatively determining the analyte.

22. The measuring apparatus as recited in claim 20 wherein the optics is a free beam optics.

23. The measuring apparatus as recited in claim 20 wherein the optics includes an optical fiber.

24. The measuring apparatus as recited in claim 23 wherein the sensor is arranged at an inner side of a wall of a sample container, and the optical fiber ends at an outer side of the wall of the sample container, so that the light guided in the optical fiber propagates through the wall of the sample container.
